(19) United States
(12) Patent Application Publication
(10) Pub. No.: US 2003/0148362 A1
(43) Pub. Date: Aug. 7, 2003

(54) Diagnostic microarray and method of use thereof

(75) Inventor: Janos Luka, Virginia Beach, VA (US)

Correspondence Address: FOLEY & LARDNER, 777 EAST WISCONSIN AVENUE, SUITE 3800, MILWAUKEE, WI, 53202-5308, US

(73) Assignee: Eastern Virginia Medical School of the Medical College of Hampton Roads

(21) Appl. No.: 10/357,756

(22) Filed: Feb. 4, 2003

Related U.S. Application Data
(60) Provisional application No. 60/355,460, filed on Feb. 7, 2002.

(51) Int. Cl. C12Q 1/68; C12M 1/34

(52) U.S. Cl. 435/6; 435/287.2; 205/777.5

(57) ABSTRACT

A microarray device for the analysis of biological samples is provided. The device includes a liquid permeable layer having a plurality of microregions, each including a plurality of probe-labeled microbeads embedded in the liquid permeable layer. The microbeads in a given microregion include a plurality of the same target probes on their surfaces. The target probes are capable of specifically binding to one or more particular target molecules (e.g., nucleic acid, polypeptide, small molecule antigen). The device typically has the capability of inducing a sample solution to move through the liquid permeable layer under the influence of an applied voltage. Kits which include the device and methods of simultaneously detecting a plurality of different target molecules in a sample solution are also provided.

### CROSS-REFERENCE TO PROVISIONAL APPLICATION

This application claims the benefit of U.S. Provisional Patent Application Serial No. 60/355,460, filed Feb. 7, 2002, entitled: Diagnostic Microarray And Method Of Use Thereof, which is incorporated herein by reference.

### BACKGROUND

Molecular biology comprises a wide variety of techniques for the analysis of nucleic acids and proteins, many of which form the basis of clinical diagnostic assays. These techniques include nucleic acid hybridization analysis, restriction enzyme analysis, genetic sequence analysis, and separation and purification of nucleic acids and proteins. Many molecular biology techniques are, however, complex and time consuming, and generally require a high degree of attention to detail. Often such techniques are limited by a lack of sensitivity, specificity, or reproducibility.

Diagnostic assays employing directed binding of nucleic acids or proteins associated with disease offer important advantages over traditional diagnostic tests. Current tests used to diagnose an illness by the presence of antibodies are often indirect. Because indirect tests generally determine the presence of specific antibodies produced by the patient's immune system, such tests are unable to indicate whether a disorder occurred in the past or is current. Most tests are also unable to indicate whether or not there is a response to therapy. Furthermore, because it commonly takes seven to fourteen days for the immune system to mount an immune response, a diagnostic test based on the presence of antibodies can miss the recent onset of a disease. This can be especially dangerous if the ailment is capable of spreading rapidly in the patient or is easily transmitted. Employing directed complementary binding assays to detect the presence of nucleic acids or proteins directly associated with an infection or disease in a patient can give an indication of the severity and progression of the disorder.

A common method used to detect the presence of genetic material associated with an infectious agent or pathologic gene in a patient is a diagnostic assay that relies on a polymerase chain reaction (“PCR”). PCR uses an enzymatic reaction to amplify specific nucleic acid sequences from an infectious agent or pathologic gene present in a sample. PCR uses specific oligonucleotides, primers, which bind to the target nucleic acid sequences to carry out this amplification process. The nature of the PCR reaction makes it difficult to detect more than one agent simultaneously in a diagnostic assay. This makes the diagnostic use of PCR for the determination of more than one infectious agent or disease molecule, costly and labor intensive.

A variety of devices have been designed and fabricated to actively carry out and control directed complementary binding reactions in microscopic formats. These binding reactions can include nucleic acid hybridization, antibody/antigen associations, and similar reactions. Such devices have been fabricated using microlithographic and micro-machining techniques. These devices are reported to be able to remove non-specifically bound molecules, provide stringency control for binding reactions, and improve the detection of analytes. These devices commonly rely upon the binding of a target molecule with a complementary probe. Assays using these devices are often required to detect very low concentrations of specific target molecules (DNA, RNA, antibodies, receptors, etc.) from among a large amount of non-target molecules that can have very similar composition and structure. Binding reactions are normally carried out under the most stringent conditions, achieved through various combinations of temperature, salts, detergents, solvents, chaotropic agents, and denaturants to ensure specificity.

Assays employing directed complementary binding reactions offer the promise of improved diagnostic tests for the detection of different genetic disorders and infectious agents. Microarrays, in particular, show great potential as diagnostic tests because of their ability to detect the presence of a large number of different target molecules in a single experiment. Many of the current microarrays do not meet the requirements necessary for the optimal use as a diagnostic assay. The current microarray formats and stringency control methods are often unable to detect low copy number (i.e., 1-100,000) biological targets even with the most sensitive reporter groups (enzyme, fluorophores, radioisotopes, etc.) and associated detection systems (fluorometers, luminometers, photon counters, scintillation counters, etc.). Current techniques may require very high levels of relatively short single-stranded target sequences or PCR amplified DNA, and can produce a high level of false positive hybridization signals even under the most stringent conditions. In addition, many of the current hybridization assays are not quantitative and can be subject to substantial variability. Results between studies using microarrays often show poor comparability.

These problems are all associated, in one way or another, with the unfavorable binding dynamics between a complementary binding probe and its specific target. A common problem with diagnostic assays is that the concentration of a target molecule in a biological sample is often very low. In addition, a probe often has to compete with the complementary strand of the target nucleic acid that is normally present along with the target molecule in a biological sample. Binding reactions are concentration and time dependent. A decrease in the concentration of the target molecule will decrease the efficiency and the rate of the binding of the target to its complementary probe.

Furthermore, the surface area of the microregion limits the amount of probe that can be deposited in a microregion. In addition, there are often variations in the amount of probe bound to a specific microregion. Similarly, there are variations in the amount of probe bound in such a way that it is accessible to hybridization of its substrate. Even small variations in the amount of probe capable of binding the target molecule in a given microregion can lead to a dramatic increase in the variability and lack of comparability of microarray results. One way to increase the sensitivity and decrease the variability of a diagnostic microarray device is to increase the amount of probe deposited in a given microregion.

Another characteristic that may limit the use of current microarrays for diagnostic applications is the cost and time required for an assay. There is a continuing need for medical diagnostic tests for infectious and genetic diseases that are accurate, cheap, convenient, and easy to use.

### SUMMARY

This invention relates to methods and devices for the analysis of biological samples for diagnostic and/or laboratory purposes and, more particularly, pertains to the design, fabrication, and uses of a device including a diagnostic microarray that is capable of carrying out diagnostic determinations in microscopic formats. The diagnostic determinations generally include complementary molecular biological reactions, such as nucleic acid hybridization or protein binding interactions. The methods can utilize a microarray that can be used to quantitate the presence of more than one pathologic gene, mRNA, and/or protein in a sample at the same time. The microarray devices described herein can provide a diagnostic test that is convenient and easy to use.

The present microarray-based diagnostic assay utilizes a device that includes a liquid permeable layer with a plurality of probe-labeled microregions. Each probe-labeled microregion includes a plurality of probe labeled microbeads embedded within the permeable layer, thereby increasing the surface area available for probes to be present within the microregion. The surfaces of the probe labeled microbeads within a given microregion include a plurality of probes which are capable of specifically binding to a particular target molecule (e.g., nucleic acid, polypeptide, small molecule antigen). In most instances, the device includes a plurality of different probes where the microbeads in each microregion contain identical probes on their surfaces. The device also generally includes two liquid chambers, each containing an electrode, in fluid connection with the permeable layer. This provides the device with the capability of inducing a sample to move through the liquid permeable layer under the influence of an applied voltage.

A sample can be introduced into one of the liquid chambers and induced to move through the liquid permeable layer by applying a voltage across the electrodes in the two chambers, i.e., electrophoretically transporting the sample solution through the liquid permeable layer. As the sample passes through the liquid permeable layer, the “target probes” on the microbeads bind target molecules. The bound target molecules can be detected by a variety of conventional techniques, e.g., the displacement of visualization probes, such as fluorescent-labeled target molecules, via competitive binding by the target molecules or binding of visualization probes which are capable of specifically recognizing a particular target probe/target molecule complex. The present method can permit the detection of extremely small quantities of specific target molecules in a sample, e.g., the detection of the presence of as little as 500 copies of a nucleic acid or protein in a sample, without necessitating the use of amplification techniques such as PCR.

Another embodiment is directed to a method of production of the diagnostic microarray. The probe-labeled microbeads can be introduced to a microregion on a solid support in a suspension in a viscous liquid permeable medium. The solid support is commonly formed from an electrically non-conducting material, such as plastic, glass or other non-conducting ceramic material. For example, a suspension of probe-labeled microspheres having a diameter of about 20 to 500 nm can be suspended in a matrix solution, e.g., a 0.1-2.0 wt. % aqueous agarose solution. The suspension of the microspheres can then be introduced in drop form onto microregions (e.g., having a diameter of about 5 to 10 microns) of a solid support, such as a glass or plastic slide. The drops are typically allowed to solidify and then covered with a thin layer (e.g., 5-20 microns thick) of a matrix solution. One example of a suitable matrix solution is a solution of agarose in an appropriate electrophoresis buffer (e.g., about 0.3 to 1.0 wt. % agarose solution).

A kit that includes the diagnostic microarray device and a zwitterionic electrophoresis buffer is also provided herein. The buffer is desirably selected to enhance the binding rate of the target molecule and complementary probe. The kit commonly also includes visualization probes (e.g., fluorescent-labeled probes or enzyme-labeled probes). For example, the visualization probes may be capable of recognizing the presence of a complementary pair formed by the binding of a target molecule with its complementary probe. Other suitable visualization probes include fluorescent- or enzyme-labeled forms of (a) the target molecule, (b) an appropriate fragment of the target molecule or (c) a closely related analog of the target molecule. This latter type of visualization probe can be used to detect the presence of target molecules in a sample via a competitive binding assay.

A number of illustrative embodiments of the present diagnostic microarray devices and methods that employ the device(s) are described herein. The embodiments described are intended to provide illustrative examples of the present microarray devices and related methods and are not intended to limit the scope of the invention.

### BRIEF DESCRIPTION OF THE DRAWINGS

FIG. 1 shows a top view of one embodiment of the present diagnostic microarray device.

FIG. 2 shows a cross-sectional view of the diagnostic microarray device shown in FIG. 1, with positive and negative electrodes inserted into the buffer chambers.

FIG. 3 shows a top view of an embodiment of the present diagnostic microarray device that is capable of simultaneously conducting analyses of three different samples, e.g., an unknown sample and two different standard samples.

FIG. 4 shows a schematic representation of positive and negative analysis for two different microspheres containing specific probes on their surfaces.

FIG. 5 shows a graph depicting the results of analyses for copy numbers of a nucleotide sequence associated with HIV in blood samples using the present method versus those obtained with a PCR based method.

FIG. 6 depicts fluorescence analysis of microarray analysis of blood samples from four AIDS patients for the presence of nucleotides associated with fourteen different infectious agents.

### DETAILED DESCRIPTION

A microarray device for the analysis of biological samples is provided. The device includes a liquid permeable layer including a plurality of probe-labeled microbeads embedded in the liquid permeable layer. The microbeads in a given microregion typically include a plurality of the same target probes on their surfaces. The target probes are capable of specifically binding to one or more particular target molecules (e.g., nucleic acid, polypeptide, small molecule antigen). The device commonly has the capability of inducing a sample solution to move through the liquid permeable layer under the influence of an applied voltage. The microarray device takes advantage of directed complementary binding reactions to offer improved diagnostic tests for the detection of different genetic disorders and infectious agents. The microarray device can offer great potential as a diagnostic test because it can permit the simultaneous rapid detection of the presence of a large number of different target molecules in a single experiment. Kits which include the device and methods of simultaneously detecting a plurality of different target molecules in a sample solution are also provided.

The current device may be created by first introducing target probes onto surfaces of a lot of microbeads, such as surfaces of pre-activated microspheres. As employed herein, the term “lot” refers to microbeads which have the same target probes present on their surfaces. Commonly, the microbeads in a given lot all have a plurality of a single target probe on their surfaces. In some circumstances, however, it may be useful for all of the microbeads in a given lot to have a plurality of two (or more) different target probes on their surfaces. Whether a single type of two or more different target probes are present on the surfaces of microbeads in a given lot, it is generally preferable to have the various target probes present at the same relative concentrations on the surfaces of microbeads in the lot.

The microbeads are commonly then suspended in a liquid permeable matrix, which can be formed from a material, such as agarose, polyacrylamide, cellulose or gelatin. The suspension of the beads can be distributed onto specific portions of a surface (“microregions”). The microbeads typically are deposited as a suspension in a flowable form of a liquid permeable medium in discrete microregions on the surface. The deposited suspension is commonly allowed to solidify and then covered with an additional liquid permeable material to form the liquid permeable layer. Following this, the chip can then be put into an apparatus with two buffer chambers (each having an electrode therein) at opposite ends of the chip. Buffer can be added so that it is in contact with the permeable layer of the chip and can complete an electric circuit when current is supplied to the device.

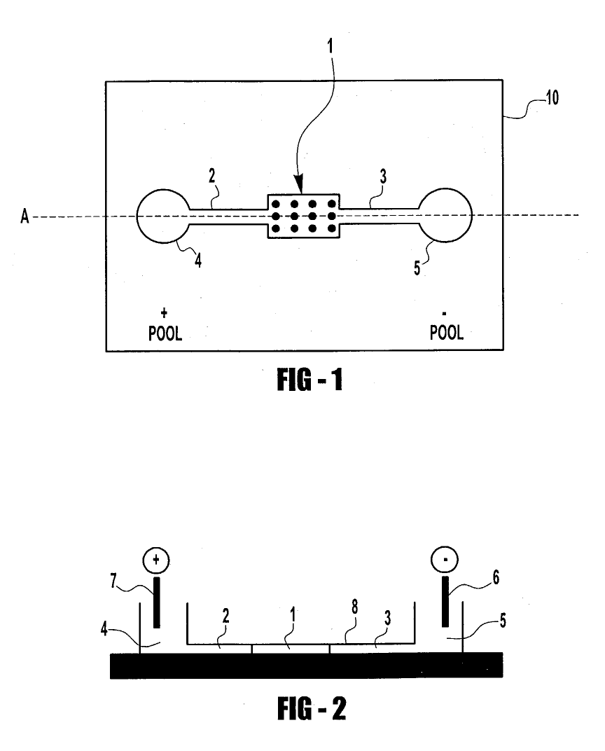

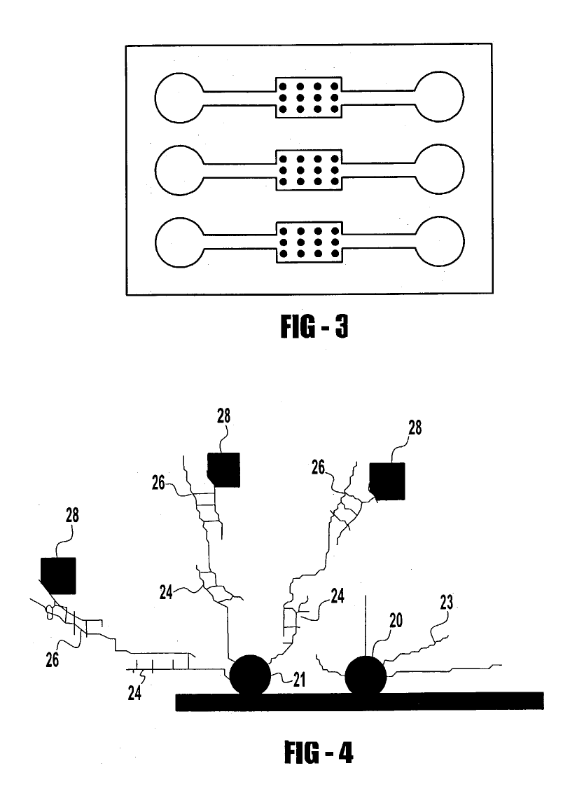

FIGS. 1 and 2 depict one example of the present microarray device. FIG. 1 shows a top view of the device 10 which includes a liquid permeable layer 1 containing twelve microregions with probe-labeled microbeads embedded in the liquid permeable matrix. Liquid permeable layer 1 is connected to buffer chambers 5 and 4 (“liquid chambers”) by fluid channels 2 and 3. FIG. 2 shows a cross-sectional view of the microarray device (along line A of FIG. 1) with electrodes 6 and 7 inserted into the buffer chambers 5 and 4. The cross-sectional view shows a cover slide 8 covering the liquid permeable layer 1 and connecting fluid channels 2 and 3. FIG. 3 shows a top view of an alternate embodiment of the present microarray device. The device depicted in FIG. 3 includes three sets of liquid permeable layers connected to buffer chambers via fluid channels and thus is capable of simultaneously conducting analyses of three different samples, e.g., an unknown sample and two different standard samples.

In one embodiment, a microarray device for the analysis of biological samples is provided. The device includes a liquid permeable layer including a plurality of microregions. Each microregion includes a plurality of probe-labeled microspheres embedded in the liquid permeable layer. All of the microspheres in a given microregion have a plurality of the same target probes on their surfaces. Typically, the microspheres in a given microregion will have probes for a single target molecule on their surfaces. In some instances, however, it may be desirable to have more than one type of probe on the surface of microbeads in a given microregion. This can be accomplished by depositing two different sets of microbeads, each labeled with a different probe, in a single microregion, i.e., microbeads from two different lots may be deposited in a single microregion. This can also be accomplished by introducing two different probes onto the surfaces of all of the microbeads deposited in a single microregion.

The present microarrays commonly will have microbeads labeled with a unique probe deposited in each microregion. In such an embodiment, a positive signal for a given microregion thus implies the presence (which can be determined quantitatively if desired) for the corresponding target molecule in the sample. In some instances, e.g., to provide enhanced reliability, it may be desirable to deposit microbeads labeled with probes for a given target molecule in more than one microregion.

The liquid permeable layer can be formed from a liquid permeable material such as agarose, polyacrylamide, cellulose or gelatin. For example, the liquid permeable layer may include about 0.3 to 1.0 wt. % agarose, typically in a suitable electrophoresis buffer. The liquid permeable layer generally has a relatively small volume, e.g., has a capacity to hold about 100 to 200 microliters of water or buffer solution. In order to minimize the liquid capacity and thereby minimize the amount of sample material required for an analysis, the liquid permeable layer commonly has a thickness of no more than about 50 microns and liquid permeable layers with a thickness of about 5 to 20 microns are quite suitable. The present device allows a large number of microregions to be created on a relatively small surface area. Hence, devices with small surface areas correspondingly require very small sample volumes yet are capable of being used to simultaneously analyze a large number of diseases and/or conditions can be produced using the present methods. For example, the present devices can include a liquid permeable layer which has a microregion density of about 250 to 2500 microregions per mm². Very commonly, the microregions have a largest dimension (e.g., diameter) of no more than about 10 microns.

In addition to the liquid permeable layer, the present microarray device generally includes at least one liquid chamber (“first liquid chamber”) in fluid connection with the liquid permeable layer. The first liquid chamber typically includes an electrode or is configured to receive an electrode. Very commonly, the microarray device also includes a second liquid chamber in fluid connection with the liquid permeable layer. The second liquid chamber includes typically also an electrode in fluid connection with the liquid permeable layer.

As used herein, the term “microbead” encompasses any type of solid or hollow sphere, ball, bearing, cylinder, or other similar configuration composed of ceramic, metal, and/or polymeric material onto which a target probe can be immobilized. Typically, a microbead that is spherical (“microsphere”), in shape is employed in the present devices. The microarray device typically includes microbeads that have a largest dimension of about 20 nm to 1 micron, or more suitably about 50 to 200 nm. Where the microbeads are substantially spherical in shape, the microbeads commonly have a diameter of about 20 to 500 nm and, more suitably, about 50 to 200 nm. Very often, it may be suitable to use microbeads that are unpolished or, if polished, roughened before use.

The microbeads are typically comprised of a polymeric material containing derivatizable functional groups (e.g., p-aminostyrene polymers and copolymers,and cyanuric chloride activated cellulose) or polymeric material that can be activated (e.g., nylon beads). Examples of particularly suitable materials which can be used to form the microbeads include nylon, polystyrene, glass, polypropylenes, polystyrene/glycidyl methacrylate latex beads, latex beads containing amino, carboxyl, sulfonic and/or hydroxyl groups, polystyrene coated magnetic beads containing amino and/or carboxylate groups, teflon, and the like.

In one embodiment, the microarray device comprises a set of at least about 10 different lots of probe-labeled microbeads, each different lot of probe-labeled microbeads being present in at least one separate microregion. More commonly, the microarray device can include a significantly larger number of lots of distinct lots of probe-labeled microbeads, e.g., 100 to 1,000 distinct lots of probe-labeled microbeads, each deposited in at least one separate microregion of the device.

Target probes can be covalently bound to the surfaces of the microbeads. For example, the target probes may be bound to a microsphere surface through a linker molecule. The microsphere can include at least one target probe that is a peptide. For example, the target probe may be capable of specifically binding to a protein target molecule. Suitable examples of such target probes include an antibody Fab fragment or a molecule which includes the Fab fragment (e.g., a complete antibody or a fusion protein which includes the Fab fragment) is suitable as a target probe. Alternatively, the microsphere can include at least one target probe that is a nucleic acid or an analog which is capable of binding to a nucleic acid. The nucleic acid target probe can include DNA molecules, RNA molecules, oligonucleotides containing RNA and DNA, oligonucleotides containing modified nucleotides and oligonucleotides containing protein nucleic acids.

The target probes employed in the present devices are often capable of specifically binding to a nucleic acid target molecule such as a RNA target molecule or a DNA target molecules. An example of a representative target probe is a target probe able to specifically bind a single nucleic acid target molecule selected from the group consisting of a nucleic acid sequence(s) associated with a pathogenic protein, a viral nucleic acid sequence, a bacterial nucleic acid sequence, a parasite nucleic acid sequence, a cancer specific nucleic acid sequence or a nucleic acid sequence associated with a genetic disorder. Specific examples include target probes capable of specifically binding to a nucleic acid associated with human immunodeficiency virus (“HIV”), human herpesvirus (“HHV”), herpes simplex virus (“HSV”), Epstein-barr virus (“EBV”), hepatitis C virus (“HCV”), cytomegalo virus (“CMV”), Varicella Zoster virus (“VZV”), human papiloma virus (“HPV”), Chlamydia (“Chl”), parvovirus B19 (“B19”), or a human gene (“Hu”). Particularly useful target probes which can be used in the present device include oligonucleotides capable of specifically binding to a nucleic acid target molecule from at least one of human herpesvirus 6 (“HHV-6”), human herpesvirus 7 (“HHV-7”) and human herpesvirus 8 (“HHV-8”).

Target probes employed in the present devices may be capable of specifically binding to a polypeptide or small organic molecule. Non-limiting examples of such target probes include antibodies, antigens, ligands, and receptor proteins. For example, the target molecule may be a polypeptide which includes an antibody Fab fragment, e.g., a complete antibody, a humanized antibody or a fusion protein which includes the Fab fragment.

One embodiment is directed to a method of identifying the presence of target molecules in a sample solution. The method can include:

(a) electrophoretically transporting the sample solution through a liquid permeable layer, wherein the liquid permeable layer includes at least one microregion having a plurality of labeled microbeads embedded in the liquid permeable layer; the labeled microbeads having a plurality of the target probes on their surfaces; whereby said target molecules are bound to the target probes to form probe/target complexes;

(b) electrophoretically transporting a probe solution including visualization probes through the liquid permeable layer such that the visualization probes bind to probe/target complexes to form bound visualization probes; and

(c) detecting the bound visualization probes.

Another embodiment provides a method of identifying the presence of target molecules in a sample solution which includes:

(a) electrophoretically transporting the sample solution through a liquid permeable layer, wherein the liquid permeable layer includes at least one microregion having a plurality of labeled microbeads embedded in the liquid permeable layer; the labeled microbeads having a plurality of the target probes on their surfaces; whereby the target molecules are bound to the target probes to form probe/target complexes;

(b) electrophoretically transporting a probe solution including visualization probes through the liquid permeable layer; whereby the visualization probes are bound to the target probes to form bound visualization probes; and

(c) detecting the bound visualization probes.

In many instances, if desired, the sample solution and the probe solution can be mixed together prior to introduction into the liquid permeable layer and then transported simultaneously through the liquid permeable layer.

Another embodiment is directed to a method of identifying the presence of a target molecule and, particularly a charged target molecule, in a sample. This method can include:

(a) introducing the sample in a low conductivity buffer solution into a liquid chamber;

(b) electrophoretically transporting the sample solution through a liquid permeable layer that is in fluid connection with the liquid chamber, such that a given target molecule binds to its complementary target probe on microbeads embedded in a specific microregion of the liquid permeable layer;

(c) introducing a set of fluorescent probes in a low conductivity buffer solution into the liquid chamber;

(d) electrophoretically transporting the fluorescent probe solution through the liquid permeable layer, whereby a given target molecule binds to its complementary target probe on microbeads embedded in a specific microregion of the liquid permeable layer; and

(e) detecting binding of the fluorescent probes to their complementary target probes.

The nucleic acids are typically purified prior to introduction into the liquid chamber of the diagnostic microarray. One example of an appropriate purification procedure is described below in Example 5.

Another feature of the invention pertains to a kit for the analysis of biological samples. The kit includes a microarray device. For example, the microarray device may include a liquid permeable layer having a plurality of microregions, each microregion including a plurality of probe-labeled microspheres embedded in the liquid permeable layer. All the microspheres in a given microregion preferably have a plurality of the same target probes on their surfaces. The kit also often includes (a) a low conductivity buffer solution and (b) a buffer solution including a set of visualization probes each capable of specifically binding to one of the target probes.

Diagnostic Microarray

Probe labeled microspheres can be suspended in the liquid permeable matrix solution. The suspension may be prepared in a ratio of 3 volumes of probe labeled microspheres to 7 volumes of matrix solution. The suspension can then be deposited onto a surface of a support structure such as a glass or plastic slide. The area onto which the probe labeled microspheres is deposited is referred to herein as a microregion. The microregions typically range in size from about 5 to 20 microns. In one example of the microarray, a volume of 20-100 picoliters of the suspension may be deposited as a drop onto a surface. An ink jet printer, robot, or similar method can be used to distribute the individual drops. The drops may be allowed to solidify at room temperature and then covered with a thin layer of the liquid permeable membrane solution. The liquid permeable membrane layer may be approximately ten microns deep. The liquid permeable membrane layer may be the same liquid permeable membrane the probe labeled microspheres are suspended in. The liquid permeable membrane layer may be agarose, polyacrylamide, or any other material that can be used to make a protein or nucleic acid electrophoresis gel. The microarray can be covered by a second surface, for example, a glass slide.

Complementary Binding Pair

As used herein, the term “complementary binding pair” refers to two molecules that possess a composition or structure that allows the specific binding of a first molecule to the second molecule of the complementary pair. This binding can result from hydrophobic interactions, van der wall forces, ionic attractions, and/or hydrogen bonding, etc. Suitable examples of complementary binding pair include nucleic acid molecules that form Watson Crick base pairs, nucleic acids that form non-Watson Crick base pairs, antibody/antigen interactions, receptor/ligand interactions, and aptamer/ligand associations. As employed herein, the phrase “specific binding” refers to a binding reaction between a first molecule (target probe) and a second molecule (target molecule) that is determinative of the presence of the second molecule in a heterogeneous population of proteins, nucleic acids, other biologic molecules and/or organic molecules. Under designated assay conditions, the first molecule of the complementary pair binds to the second molecule at least two times the background in the heterogeneous population and does not significantly bind to other molecules in the sample.

In theory, either member of the complementary binding pair can be introduced onto the surfaces of microbeads and used to bind its complement. Herein, the member of a complementary binding pair that is deposited on the surfaces of microbeads is referred to as a “target probe”. Its complement, i.e., the molecule whose presence is to be assayed for in a particular sample is referred to as a “target molecule”. In other words, the target molecule is the molecule that is to be detected by the assay. The target molecule may be charged. Some examples of target molecules include RNA, DNA, antigens (e.g., peptides or other organic molecules), ligands and similar molecules.

The target probe is a molecule which is commonly bound to a solid surface (of a microbead) in such a way that it is still able to bind to the target molecule. The target probe may be a RNA, DNA, or RNA-DNA molecule. Alternatively, the target probe may be a nucleic acid probe composed partially or entirely of nucleotide analogs such as peptide nucleic acids. For example, the target probe may be an oligonucleotide of about 20-40 nucleotides in length. The target probe can also be a protein molecule such as an antibody, antigen, ligand, or receptor protein.

Microbeads

The microbeads can take a variety of forms that are convenient including beads, porous beads, crushed particles, hollow tubular shapes, shapes with planar surfaces, and the like. The microbeads may have virtually any possible structural configuration so long as the immobilized target probe remains capable of binding to the target molecule. Microbeads which are particulate matter, thereby providing increased surface area for attachment of target probes, are particularly suitable. Thus, the microbeads can have a configuration which includes microparticles, porous and impermeable microbeads, and the like.

The probe-labeled microbeads (typically microspheres) employed in the present microarray device may be formed from virtually any solid material that does not substantially interfere with the complementary binding reaction (e.g., hybridization used to detect the presence of specific oligonucleotides) that allows the formation of complementary pairs of target probes with target molecules. One type of useful matrix materials are porous (fenestrated), highly convoluted and/or rugose (e.g. controlled pore) glass. Other well-known support materials which can be used to form the microbeads include, but are not limited to, natural cellulose, modified cellulose such as nitrocellulose, polystyrene, polypropylene, polyethylene, polyvinylidene difluoride, dextran, polyacrylamide, and agarose or Sepharose. Other suitable matrix materials include paper, various glasses, ceramics, metals, and metalloids. Other examples of useful support materials which can be used to form the microbeads include polacryloylmorpholide, polyamides (such as nylon), PTFE, poly(4-methylbutene), polystyrene/latex, polymethacrylate, poly(ethylene terephthalate), rayon, poly(vinyl butyrate), polyvinylidene difluoride (PVDF), silicones, polyformaldehyde, cellulose, cellulose acetate, and the like. Preferably, the microbeads are formed from a material which is resistant to nucleic acid hybridization reagents (e.g. Tris-HCl, SSC, etc.), stable at common hybridization temperatures (e.g., 30° C. to 80° C.) and does not substantially interfere with the oligonucleotide hybridization. Materials that typically bind nucleic acids (e.g. cellulose) may be suitable, however, in a preferred embodiment, an affinity matrix composed of such materials is preferably prehybridized with a blocking nucleic acid (e.g., sperm DNA) to reduce non-specific binding.

Suitable examples of materials that can be used to form the microbeads employed in the instant devices include but are not limited to silica gel; controlled pore glass; synthetic resins such as Merrifield resin, which is chloromethylated copolystyrene-divinylbenzene(DVB) resin; Sephadex.sup.R /Sepharose.sup.R; cellulose; and the like. Particularly suitable materials for use in producing the microbeads include activated polystyrene resins, e.g., chloromethylated polystyrene resins (e.g., Merrifield resin) or tosylated polystyrene resins.

The microbead may be a pre-activated microsphere. The microbead could encompass a pre-activated microbead of about 20-500 nm in size (i.e., the average largest dimension of the microbeads is about 20-500 nm) and, more suitably, about 50 to 200 nm in size. One example of suitable microbeads are microspheres formed from polystyrene which have been preactivated to include tosyl groups on their surfaces. Pre-activated microbeads of this type are commercially available within sizes ranging from 20 nm to 1 micron.

Probe Labeled Microspheres

Oligonucleotide Probe Labeled Microspheres

As used herein, the term “labeled” refers to ionic, covalent or other attachment of a target probe onto the surface of a microbead. Suitable methods for labeling microbeads include: streptavidin- or avidin- to biotin interaction; hydrophobic interaction; magnetic interaction (e.g. using functionalized Dynabeads); polar interactions, such as “wetting” associations between two polar surfaces or between oligo/polyethylene glycol; formation of a covalent bond, such as an amide bond, disulfide bond, thioether bond, or via crosslinking agents; and via an acid-labile linker. In a particularly useful embodiment for conjugating nucleic acids to beads, a variable spacer molecule is covalently introduced between the beads and the target probe. In another preferred embodiment, the conjugation is photocleavable (e.g. streptavidin- or avidin- to biotin interaction can be cleaved by a laser).

Methods of attaching a target probe to a microbead are well known to those of skill in the art and are discussed, for example, in Brown et al. (1995) Molecular Diversity 4-12; and Rothschild et al (1996) Nucleic Acids Res. 24:351-66); S. S. Wong, “Chemistry of Protein Conjugation and Cross-Linking,” CRC Press (1991); G. T. Hermanson, “Bioconjugate Techniques,” Academic Press (1995); Lerner et al. Proc. Nat. Acad. Sci. (USA), 78: 3403-3407 (1981); Kitagawa et al. J. Biochem., 79: 233-236 (1976); PCT Publication WO 85/01051; Pochet et al. Tetrahedron. 43: 3481-3490 (1987); Schwyzer et al., Helv. Chim. Acta, 67: 1316-1327 (1984); Gait, ed. Oligonucleotide Synthesis: a Practical Approach, IRL Press, Washington D.C. (1984); Koster et al. U.S. Pat. No. 6,133,436; and Lipshutz et al. U.S. Pat. No. 6,013,440. The disclosures of the attachment methods described in these references are herein incorporated by reference.

Polypeptide or Protein Probe Labeled Microspheres

Methods for immobilizing protein molecules on a solid support are well known in the art and roughly classified as follows: i) the protein is immobilized directly on a substrate by means of adsorption or casting, ii) the protein is transferred as a thin film from the surface of liquid, e.g. Langmuir-Blodgett method (LB method), and iii) proteins are immobilized by alternate adsorption with other components.

The protein may be conjugated to the solid support by covalent or noncovalent bonds. The protein can be attached noncovalently by adsorption using methods that provide for a suitably stable and strong attachment. The protein is typically immobilized using methods well known in the art appropriate to the particular solid support, providing that the ability of the protein to bind to its target molecule is not destroyed. For a review of protein immobilization and its use in binding assays, see, for example, Butler, J. et al. In: Van Regenmortel, M. H. V., ed., Structure Of Antigens, Volume 1, CRC Press, Boca Raton, Fla., 1992, pp. 209-259, the disclosure of which is herein incorporated by reference. Immobilization may also be indirect, for example by the prior immobilization of a molecule that binds stably to the protein or to a chemical entity conjugated to the protein. For example, passive adsorption or covalent attachment may immobilize an antibody (polyclonal or monoclonal) specific for the protein. The protein is then allowed to bind to the antibody, rendering the protein immobilized. Indirect immobilization, as intended herein, includes bridging between the protein and the solid surface using any of a number of well-known agents and systems. For example, Suter, M. et al., Immunol describes the “Protein-Avidin-Biotin-Capture” (PABC) system. Lett. 13:313-317 (1986) also incorporated by reference. In such a system, passive adsorption (or covalent linking) immobilizes any biotinylated protein to the solid phase. Streptavidin, which is multivalent, binds with high affinity to the biotin sites on the immobilized protein while maintaining available binding sites for biotin in solution. The protein, in biotinylated form, is then allowed to bind to the immobilized streptavidin, rendering the protein immobile. Alternatively, the streptavidin can be passively adsorbed or covalently bound to the solid phase without the intervening protein. A protein immobilized by any of the foregoing approaches and other target probes peptides may be employed (provided that they do not interfere with its ability to bind and retain a target molecule).

Liquid Permeable Layer

The liquid permeable layer is a matrix of liquid permeable material in which probe labeled microbeads are embedded in one or more microregions. The liquid permeable layer is commonly composed of a material that is permeable to aqueous solutions and allows the flow of electrons. For example, the liquid permeable layer can be composed of a material that is used to make a nucleic acid or protein electrophoretic separation gel. The liquid permeable layer may be composed of agarose that has a concentration of 0.3% to 1% (w/v). In another example, the liquid permeable layer can be composed of polyacrylamide with a concentration of 2% to 5% (w/v). Methods of making and using the liquid permeable layer are discussed in Manniatis, Methods in Molecular Biology, vol. 3 and 4, J. M. Walker, ed., Humana Press (1984), the disclosure of which is herein incorporated by reference.

Biological Sample Preparation

Standard reference works setting forth the general principles of recombinant DNA technology and cell biology, and describing conditions for isolation and handling of nucleic acids, denaturing and annealing nucleic acids, hybridization assays, and the like, include: Sambrook, J. et al., Molecular Cloning: A Laboratory Manual, 2nd Edition, Cold Spring Harbor Press, Cold Spring Harbor, N.Y., 1989; Alberts, B. et al., Molecular Biology Of The Cell, 2nd Ed., Garland Publishing, Inc., New York, N.Y., 1989; the disclosures of which are hereby incorporated by reference in their entirety.

Biological Targets

The diagnostic microarray can be designed to detect the presence of a molecule associated with a disease or condition. This molecule, for example may be associated with a genetic disorder, toxin, or infectious agent. The infectious agents that can be analyzed by the current invention include, but are not restricted to, the Human Deficiency Virus (HIV), Human Herpes Virus-6 (HHV-6), Herpes Simplex Virus (HSV), Epstein Barr Virus (EBV), hepatitis C virus (HCV), Cytomegalovirus (CMV), Varicella-Zoster virus (VZV), Human Papilloma Virus (HPV), parvovirus B19 (B19), and Chlamydia (Chl).

Visualization Probes

The presence of target molecules bound to probes on the surfaces of microbeads can be detected by a variety of conventional techniques, e.g., the displacement of visualization probes, such as fluorescent-labeled target molecules, via competitive binding by the target molecules or binding of visualization probes which are capable of specifically recognizing a particular target molecule or a particular target probe/target molecule complex.

The present methods typically employ a visualization probe to detect the presence of target molecules bound to target probes on the surface of a microbead. The visualization probes may be capable of (a) specifically binding to a complementary target probe/target molecule complex to form a bound visualization probe; or (b) specifically binding to a target molecule. In another embodiment, the visualization probes may include labeled target molecules which are capable of specifically binding to complementary target probes to form labeled target molecule/target probe complexes.

The visualization probes may be capable of recognizing the presence of a complementary pair formed by the binding of a target molecule with its complementary probe. Other suitable visualization probes include fluorescent- or enzyme-labeled forms of (a) the target molecule, (b) an appropriate fragment of the target molecule or (c) a closely related analog of the target molecule. These latter types of visualization probe can be used to detect the presence of target molecules in a sample via a competitive binding assay.

Another type of visualization probe is capable of binding to a portion of the target molecule. This type of visualization probe is typically capable of binding to a target molecule in a manner that will not interfere with the binding of the target molecule to a complementary target probe. An example of the use of this type of probe is depicted schematically in FIG. 4. The schematic representation depicts positive and negative analysis using two different microspheres 20 and 21 containing specific probes on their surfaces. No target molecules in the sample are bound to the specific target probes 23 on the labeled microsphere on the right. The microsphere 21 on the left hand side is depicted with nucleic acid target probes 24 which are capable of hybridizing to a specific nucleic acid (e.g., a nucleic acid associated with an infectious agent such as HIV). Complementary nucleic acids 26 found in the sample (“target molecules”) are shown hybridized to the nucleic acid target probes. In the schematic representation, the sample also contains visualization probes 28 which are fluorescent labeled nucleic acids capable of hybridizing to the bound nucleic acid 26 associated with the infectious agent. The bound infectious agent associated nucleic acid can then be detected by fluorescence using established techniques.

The visualization probe can include a protein, polypeptide, or oligonucleotide that possesses a composition and structure that allows the selective attachment of the labeled probe to the target molecule or to a target molecule/target probe complex. This attachment can result from hydrophobic interactions, van der wall forces, ionic attractions, hydrogen bonding and the like. Examples of such visualization probes include receptor molecules, ligands and polypeptides which include an antibody binding domain capable of binding its complementary antibody (e.g., monoclonal antibodies and fusion proteins which include an antibody Fab fragment).

The visualization probes commonly include a detectable label, which may be conjugated to a member of a complementary binding pair. As employed herein, the term “detectable label” is intended to include not only a molecule or moiety which can be “directly” detected (e.g., a radionuclide or a chromogen) but also a moiety such as biotin, which is “indirectly” detected by its binding to a second (or third) binding partner, one of which carries the “direct” label. The labeled probe may be biotin-modified that is detectable using a detection system based on avidin or streptavidin that binds with high affinity to biotin. The avidin or streptavidin is preferably conjugated to an enzyme, the presence of which is detected by allowing the enzyme to react with a chromogenic substrate and measuring the color developed. Suitable examples of useful enzymes in the methods of the present invention are horseradish peroxidase (HRP), alkaline phosphatase, glucose-6-phosphate dehydrogenase, malate dehydrogenase, staphylococcal nuclease, delta-V-steroid isomerase, yeast alcohol dehydrogenase, alpha-glycerophosphate dehydrogenase, triose phosphate isomerase, asparaginase, glucose oxidase, beta-galactosidase, ribonuclease, urease, catalase, glucoamylase and acetylcholinesterase.

Other examples of detectable labels include: (1) a radioisotope which can be detected by such means as the use of a gamma counter or a scintillation counter or by autoradiography; (2) a fluorescent compound, which, when exposed to light of the proper wave length, becomes detectable due to its fluorescence and is measured by microscopy or fluorometry. Commonly used fluorescent labeling compounds include fluorescein isothiocyanate, rhodamine, phycoerythrin, phycocyanin, allophycocyanin, o-phthaldehyde and fluorescamine. The detectable label may be a fluorescence emitting metal such as sup 152 Eu, or others of the lanthanide series which can be attached to the oligonucleotide using metal chelating groups such as diethylenetriaminepentaacetic acid or ethylenediaminetetraacetic acid.

The detectable label may be a chemiluminescent compound, the presence of which is detected by measuring luminescence that arises during the course of a chemical reaction. Examples of useful chemiluminescent labeling compounds are luminol, isoluminol, theromatic acridinium ester, imidazole, acridinium salt, oxalate ester and ruthenium and osmium bipyridyl chelates. Likewise, a bioluminescent compound may be used to label the oligonucleotide and is detecting by measuring luminescence. In this case, a catalytic protein increases the efficiency of the chemiluminescence reaction. Examples of useful bioluminescent labeling compounds include luciferin, luciferase and aequorin.

Electrophoretic Buffers

Buffer solutions which have relatively low conductivities are typically used in conjunction with the present microdevice, particularly where the sample is to be probed for the presence of one or more nucleic acids. Examples of suitable solutions include buffers with a conductivity of about 5 to 50 μS/cm. Commonly, the low conductivity buffer has an inorganic salt content of no more than about 10 μM. The low conductivity buffer for electrophoresis of nucleic acid generally includes a zwitterion. Non-limiting examples of zwitterion amino acids include lysine and zwitterionic imidazole compounds (such as histidine). The concentration of histidine may be about 50-100 mM. The concentration of lysine is typically about 20-200 mM. Other low conductivity buffers may include a nitrogen base selected from the group consisting of tertiary amino acids and mixtures thereof. Where the buffer is designed to be utilized in the analysis of samples for the presence of specific protein molecules, the low conductivity buffer may also include a compound such as barbituric acid and substituted barbituric acids (e.g., barbital).

Zwitterionic buffers (e.g., amino acid buffers), and Tris-Borate buffers at or near their isoelectric points (“pI”) have several advantages over other types of buffers regarding the rate of electrophoretic transport and hybridization of nucleic acid. For instance, these buffers can be used at relatively high concentrations to increase buffering capacity. In addition, their conductance is commonly significantly lower than other types of buffers at the same concentration. The buffers which are used in the present method are generally a low conductivity buffer, e.g., a buffer with a conductivity of about 5 to 50 μS/cm and, more suitably about 10 to 20 μS/cm. Where the buffer is to be used in conjunction with a nucleic acid analysis, the low conductivity buffer typically also has a relatively low inorganic salt content, e.g., no more than about 10 mM.

Amino acid buffers have buffer capacity at their pI's. While a given amino acid may or may not have its “highest buffering capacity” at its pI, it will generally have some degree of buffering capacity. Buffer capacity typically decreases by a factor of 10 for every pH unit difference between the pI and the pKa. Amino acids with three ionizable groups (histidine, cysteine, lysine, glutamic acid, aspartic acid, etc.) generally have higher buffering capacities at their pI than amino acids with only two dissociations (glycine, alanine, leucine, etc.). For example, histidine pI=7.47, lysine pI=9.74, and glutamic acid pI=3.22, all have relatively good buffering capacity at their pI, relative to alanine or glycine which have relatively low buffering capacities at their pI (see A. L. Lehninger, Biochemistry, 2ed, Worth Publishers, New York, 1975; in particular FIGS. 4-8 on page 79, and FIGS. 4-9 on page 80). Histidine has been proposed as a buffer for use in gel electrophoresis, see, e.g., U.S. Pat. No. 4,936,963, but hybridization is not performed in such systems. Cysteine is in a more intermediate position, with regard to buffering capacity. The pI of cysteine is 5.02. An acid/base titration curve of 250 mM cysteine, shows that cysteine has a better “buffering capacity” at about pH 5 than a 20 mM sodium phosphate. In the pH 4 to 6 range, the buffering capacity of cysteine is significantly better than 20 mM sodium phosphate, particularly at the higher pH. However, in these pH ranges the conductance of the 250 mM cysteine solution is very low about 23 μS/cm, compared to 20 mM sodium phosphate that has a value of about 2.9 mS/cm, a factor of 100 times greater.

Several electrophoretic techniques developed over 20 years ago are based on the ability to separate proteins in zwitterionic buffers “at their pI”. These techniques are called isoelectrophoresis, isotachophoresis, and electrofocusing (see, e.g., chapters 3 and 4 in “Gel Electrophoresis of Proteins: A Practical Approach” Edited by B. D. Hames & D. Rickwood, IRL Press 1981). The use of various amino acid buffers these applications, all at their pI, are described in this reference (see, e.g., Table 2, page 168).

The present methods directed to the detection of nucleic acids typically employ buffers which can enhance the electrophoretic hybridization of nucleic acids. The buffer used for diagnostic detection of nucleic acids typically contains a zwitterion, and commonly also include a magnesium salt. The zwitterion in the buffer is commonly histidine or other ampholyte, such as a tertiary amino acid (e.g., a tertiary amino acid which is zwitterionic in the pH range of 5-7). One example of a suitable electrophoresis buffer for nucleic acid detection is a zwitterionic buffer which contains MgCl₂ (e.g., 0.001 to 0.01 M MgCl₂).

A suitable electrophoresis buffer for use in protein detection is a low conductivity buffer which includes barbituric acid and/or barbital. In these types of buffers, almost every protein migrates to the positive electrode. The inclusion of a low percentage of sodium dodecyl sulfate (“SDS”) (e.g., 0.01% SDS) can aid in maintaining relatively insoluble proteins in solution.

Electrophoretic Hybridization

Samples to be analyzed for the presence of target molecules are commonly purified prior to analysis to remove contaminants. If a purification procedure is employed, care must be taken that the procedure will not result in the removal of target molecules. For analysis of nucleic acid containing solutions, following purification, a buffer solution containing the target molecule(s) is commonly loaded into the negative electrophoretic chamber of a diagnostic microarray covered with the appropriate electrophoresis buffer. The electrodes can then be connected to the negative and the positive terminals of the power supply. A current of about 10-100 microamperes is typically applied to the microarray for about 2-10 minutes, e.g., a current of about 60-90 microamperes may suitably be applied to microarrays where the liquid permeable layer is about 5 to 20 microns in thickness.

Following electrophoretic transport of the sample solution through the device, the microarray can be analyzed for the presence of target molecules bound to target probes on the surfaces of microbeads using standard techniques. In one exemplary embodiment, purified nucleic acid from a sample may be denatured and mixed with complementary nucleic acid probe labeled with fluorescent tag. The mixture can then be introduced into the chamber that is connected to the negative electrode of a power supply. A low power (e.g., 50 to 100 microamperes) electric field can be applied to the device for a relatively short period of time, e.g., for about 5 to 20 minutes. The microarray is commonly analyzed for a probe signal using a fluorescence image analyzer.

Another procedure includes loading a solution containing the target molecule into the microarray following purification. The electrophoretic procedure described above can then be performed. A buffer solution containing appropriate visualization probes can then be loaded into the microarray and the electrophoresis step can be repeated. The microarray can then be analyzed as in the previous procedure for binding of the visualization probe, e.g., either via competitive binding to a target probe or binding to a target probe/target molecule complex.

### EXAMPLES

The following examples are presented to illustrate the present invention and to assist one of ordinary skill in making and using the same. The examples are not intended in any way to otherwise limit the scope of the invention.

### Example 1

### Coupling of Nucleic Acids to Beads

Pre-activated microbeads (e.g., tosyl activated) formed from polystyrene (80 nm±3% size) are mixed in phosphate buffer with oligonucleotide probes which have been 5′-amino modified via a 12 carbon linker. The probes are typically circa 25-40 nucleotides in length. The probes are selected to correspond to the complement of a target nucleotide to be detected. The mixture of pre-activated microbeads and 5′-amino modified oligonucleotide probes is allowed to react at +4° C. for 16-20 hours. The beads are then washed with 1 M ethyleneamine buffer and blocked with bovine serum albumin in phosphate buffer for 2 hours. After a final wash with phosphate buffer, the beads can be stored in phosphate buffered saline (“PBS”) with a preservative (e.g., sodium azide) at +4° C. for a year or longer.

### Example 2

### Coupling of Peptides to Beads

Tosyl pre-activated microbeads (80 nm) are mixed in phosphate buffer with peptide probe molecules. Depending on the type of assay to be conducted, either antibodies (or related Fab fragments) and/or antigenic peptides can be employed as the probe molecules. The resulting mixture is allowed to react at +4° C. for 16-20 hours. The probe-modified beads are then washed with 1 M ethyleneamine buffer and blocked with bovine serum albumin in phosphate buffer for 2 hours. After a final wash with phosphate buffer, the beads can be stored in PBS with a preservative at +4° C. for one year or longer.

### Example 3

### Deposition of Labeled Beads on a Support

A matrix solution of probe-labeled microbeads in 0.5 wt. % agarose at 40° C. is prepared to give a solution in electrophoresis buffer with a final composition of about 30 wt. % microbeads per unit weight of agarose. Suitable electrophoresis buffers are described below. Microbeads coupled to nucleic acid or protein probe are resuspended in the matrix solution at 40° C. in a ratio of 3 volumes of beads to 7 volumes of matrix solution. A volume of about 20-100 picoliters of the suspension is distributed as a drop onto a glass surface in a humid environment to avoid drying of the sample. An ink jet printer, microarray robot, or other similar device can be used to deposit the individual drops. The drops are allowed to solidify at room temperature and then covered with a thin layer (approximately 10 microns) of a desired matrix material (e.g., 0.5 wt. % agarose in the corresponding electrophoresis buffer). The matrix solution generally contains the same material (e.g., agarose) and electrophoresis buffer as the material used to form the suspension of microbeads.

The electrophoresis buffer used for nucleic acid detection may be composed of a zwitterionic buffer (e.g., histidine buffer) containing 0.01M MgCl₂. The inclusion of MgCl₂ in the electrophoresis buffer can increase the hybridization efficiency of the nucleic acids.

The electrophoresis buffer for protein detection may be composed of a barbituric acid or barbital buffer containing 0.01% SDS. The SDS in the electrophoresis buffer can allow proteins which are insoluble under normal conditions to stay in solution, thereby allowing such proteins to be more readily detected. In buffers of this type, almost all protein migrate to the positive electrode under electrophoretic conditions.

### Example 4

### Construction of a Microarray Device

A glass slide with microregions of microbeads embedded in a suitable liquid permeable can be covered with a glass cover slip. The opposite ends of the resulting array can be connected to liquid chambers. The chambers are capable of being filled with electrophoresis buffer containing a sample and/or visualization probe or simply with buffer. Prefabricated “chips” of this type can be sealed and stored at +4° C. for prolonged periods of time.

### Example 5

### Purification of Nucleic Acids Prior to Analysis

A tissue and/or fluid sample (e.g., blood, urine, and the like) is suspended in 10 volumes of a solution which contains either 6 M guanidine-thiocyanate or 6 M guanidine-HCl. After incubation for 5 minutes at room temperature, a 50 wt. % mixture of silica powder in deionized water is added to the sample solution and the resulting mixture is vortexed for 30 seconds. The mixture is then incubated for an additional 3 minutes at room temperature. The silica is then sedimented via centrifugation and washed with 10 mM tris-HCl pH 7.4 containing 50% ethanol. The silica is washed a second time with the same buffer. Bound DNA is then eluted from the washed silica at about 95° C. using 100 μl of an electrophoresis buffer suitable for nucleic acid detection. The eluted nucleic acids are ready to be loaded into the electrophoresis chamber of the present microarray device. Alternatively, the silica containing the bound DNA can be mixed with electrophoresis buffer, heated to about 95° C. for one minute and the resulting slurry loaded directly into the chamber. The bound DNA will elute under the application of electrophoresis.

### Example 6

### Microassay of Fluid Sample for Specific DNA

A nucleic acid sample purified according to Example 5 is mixed with the fluorescence labeled oligonucleotide probes (typically circa 25-40 nucleotides in length). The resulting mixture is loaded into the negative electrophoretic chamber of the present microarray device. The electrodes are connected to the negative and the positive pool of the power supply and a power is applied (typically 10-50 microamperes). After about 5 minutes the power is disconnected and the microchip is analyzed for fluorescent signals by an image analyzer. Fluorescent signals from the sample are compared with signals from known amount of standards run simultaneously. The concentration of target oligonucleotides in the purified nucleic acid sample can be calculated from the decrease in signal due to competitive binding of the target oligonucleotides versus the fluorescence labeled oligonucleotide probes.

### Example 7

### Detection of HIV-Related DNA in Blood

A microarray device was created which had a microregion containing microbeads coupled to an oligonucleotide probe complementary to a nucleotide sequence from the Human Immunodeficiency Virus gag gene. Plasma samples from 86 AIDS patients were purified according to the procedure described in Example 5. The purified plasma samples were quantitatively assayed for the presence of HIV RNA by the present microarray-based method. The assay was conducted using samples eluted with a histidine buffer (50 M histidine) containing 0.01M MgCl₂. The samples mixtures were loaded into the negative electrophoretic chamber of a microarray device and 30 microamperes power was applied for 3 minutes. After about 5 minutes, a solution of fluorescence labeled probes including a nucleotide sequence from the Human Immunodeficiency Virus gag gene in the electrophoresis buffer were introduced to the negative electrophoretic chamber of a microarray device. The probe solution was electrophoretically transported through the microarray device by applying 30 microamperes across the electrodes for 3 minutes. The concentration of target oligonucleotides in the purified nucleic acid sample was calculated from the decrease in signal due to competitive binding of the target oligonucleotides versus the fluorescent labeled oligonucleotide probes. Fluorescent signals from simultaneously run, standard samples having known concentrations of the target oligonucleotides were used to calibrate the results.

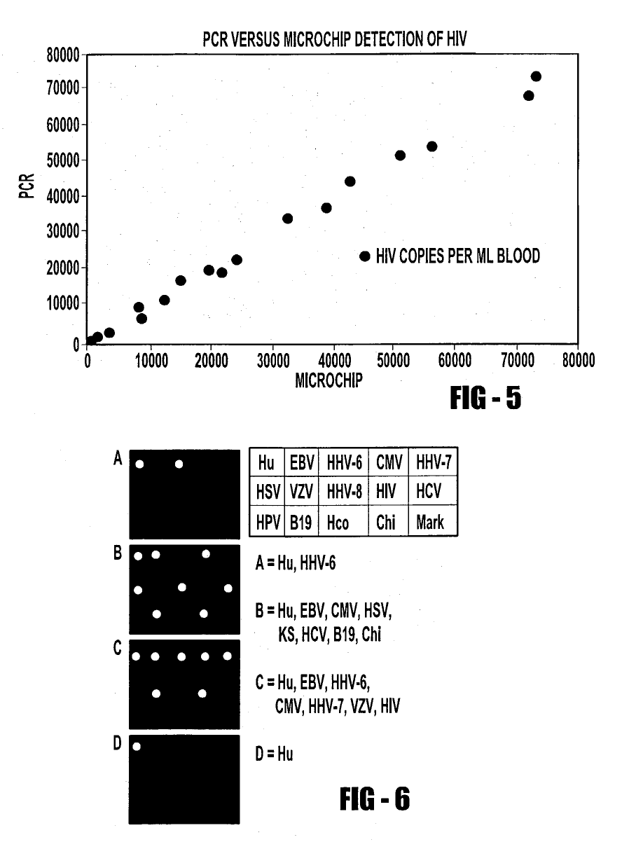

For comparison purposes, the samples were also assayed by a standard PCR-based method. The PCR assay was carried out using the QA-RT-PCR method which has been described in Dumont et al., Blood, vol. 97, 3640-3647 (2001). The viral copy numbers in the patient samples varied from about 100 to 75,000 copies per 0.1 mL of plasma. FIG. 5 shows a comparison of the results obtained via the standard PCR procedure versus those obtained using the present microarray-based method. As the graph demonstrates, the data show close to a linear correlation between the results obtained by the two methods over a wide range of concentrations of the target RNA (100 to 75,000 copies per 0.1 mL of plasma).

### Example 8

### Detection of Multiple Pathogen Markers in Blood Samples

Oligonucleotides corresponding to complementary sequences to nucleotide sequences associated with 14 different infectious agents were coupled to individual batches of tosyl pre-activated 5 micron microspheres according to the procedure described in Example 2. The probes chosen were complementary to DNA sequences associated with EBV, HIV, HHV-6, HHV-7, HHV-8, HSV, HCV, CMV, VZV, HPV, Hu, B19, Eco and Chl. The microbeads in agarose (0.5 wt. % agarose in a histidine buffer (50 mM histidine) containing 0.01M MgCl₂ and 0.01% SDS) were placed on 2×2 mm glass slides using a micromanipulator. Sufficient agarose to provide a 10 micron thick liquid permeable layer was introduced onto the slides. Samples of material purified from patient's plasma was introduced onto the microarray and electrophoretically transported through the microarray device (75 microamperes for 5 minutes). After about 10 minutes, a probe solution containing fluorescent-labeled oligonucleotide probes in electrophoresis buffer was introduced to the negative electrophoretic chamber of the device. The probe solution was electrophoretically transported through the microarray device and concentration of target oligonucleotides in the purified nucleic acid sample was calculated from the decrease in signal due from the fluorescent labeled oligonucleotide probes. For comparison purposes, the samples were assayed for the same set of 14 infectious agents. The results are shown in Table I below and in FIG. 5. FIG. 6 shows the fluorescence analysis for the presence of nucleotides associated with fourteen different infectious agents of microarrays exposed to blood samples from four AIDS patients. The spot in the upper left hand corner is a control microregion. Table I lists the copy numbers for the infectious agents identified in the corresponding samples calculated from measurement of fluorescence intensity in the microregion containing the corresponding probe-labeled microbeads.

The data shows a strong correlation between the PCR method and the present fluorescent labeled oligonucleotide based-probe. To date, the methods have been employed to provide baseline data in another 130 patients infected with AIDS as well as 60 healthy blood donors. A strong correlation between these additional microarray and PCR results was observed.

TABLE I
Viral Copy Numbers by Microdevice vs. PCR

| Patient | Copy No. by Microdevice | | Copy No. by PCR | |
|---|---|---|---|---|
| A | HHV-6 = | 2,900/ml | HHV-6 = | 2,900/ml |
| B | EBV = | 5,200/ml | HHV-6 = | 2,900/ml |
| | CMV = | 1,700/ml | CMV = | 1,100/ml |
| | HSV = | 6,400/ml | HSV = | 4,800/ml |
| | KS = | 700/ml | KS = | 200/ml |
| | HCV = | 10,600/ml | HCV = | 18,500/ml |
| | B19 = | 62,500/ml | B19 = | 53,500/ml |
| | Chl = | 24,000/ml | Chl = | 29,400/ml |
| C | EBV = | 21,700/ml | EBV = | 19,100/ml |
| | CMV = | 3,100/ml | CMV = | 3,800/ml |
| | HSV = | 6,400/ml | HSV = | 4,800/ml |
| | KS = | 700/ml | KS = | 200/ml |
| | HCV = | 10,600/ml | HCV = | 18,500/ml |
| | B19 = | 62,500/ml | B19 = | 53,500/ml |
| | Chl = | 24,000/ml | Chl = | 29,400/ml |

| | | |
|---|---|---|
| D | Negative (<500/ml) | Negative (<100/ml) |

The invention has been described with reference to various specific and illustrative embodiments and techniques. However, it should be understood that many variations and modifications may be made while remaining within the spirit and scope of the invention.

We claim:

1. A microarray device for the analysis of biological samples comprising:
a liquid permeable layer including a plurality of microregions, each microregion including a plurality of microbeads embedded in the liquid permeable layer; wherein the microbeads in a given microregion have a plurality of target probes on their surfaces.

2. The device of claim 1 wherein all the microbeads in a given microregion have a plurality of a single target probe on their surfaces.

3. The device of claim 1 wherein the liquid permeable layer comprises agarose, polyacrylamide, cellulose or gelatin.

4. The device of claim 3 wherein the liquid permeable layer comprises about 0.1 to 2.0 wt. % agarose.

5. The device of claim 1 wherein the microregions have a largest dimension of no more than about 10 microns.

6. The device of claim 1 wherein the liquid permeable layer comprises about 250 to 2500 of the microregions per mm².

7. The device of claim 1 further comprising a first liquid chamber in fluid connection with the liquid permeable layer; wherein the first liquid chamber includes an electrode.

8. The device of claim 7 further comprising a second liquid chamber in fluid connection with the liquid permeable layer; wherein the second liquid chamber includes an electrode.

9. The device of claim 1 comprising a set of at least about 10 different lots of probe-labeled microbeads, each different lot of probe-labeled microbeads being present in at least one separate microregion; wherein all the microbeads in a given lot have the same target probes on their surfaces.

10. The device of claim 1 wherein the target probes are covalently bound to the surfaces of the microbeads.

11. The device of claim 1 wherein the target probes include at least one target probe which is a polypeptide.

12. The device of claim 11 wherein the polypeptide includes an antibody Fab fragment.

13. The device of claim 1 wherein the target probes include at least one nucleic acid probe capable of specifically binding to a target nucleic acid.

14. The device of claim 13 wherein the nucleic acid probe is a DNA molecule.

15. The device of claim 13 wherein the nucleic acid probe is a modified nucleotide.

16. The device of claim 13 wherein the target probes include oligonucleotides capable of specifically binding to a nucleic acid from at least one of HIV, HHV, HSV, EBV, HCV, CMV, VZV, HPV, Hu, B19, and Ch1.

17. The device of claim 13 wherein the target probes include at least one probe selected from the group consisting of oligonucleotides capable of specifically binding to a nucleic acid from at least one of HHV-6, HHV-7 or HHV-8.

18. The device of claim 1 wherein the target probes include at least one probe capable of specifically binding to a target polypeptide.

19. The device of claim 1 wherein the liquid permeable layer has a volume of about 100 to 200 microliters.

20. The device of claim 1 wherein the liquid permeable layer has a thickness of about 5 to 20 microns.

21. The device of claim 1 wherein the microbeads are about 50 to 200 nm in size.

22. A method of detecting one or more target molecules in a sample solution, the method comprising:
(a) electrophoretically transporting the sample solution through a liquid permeable layer, wherein the liquid permeable layer includes at least one microregion having a plurality of microbeads embedded in the liquid permeable layer; the microbeads having a plurality of target probes on their surfaces; wherein the target probes are capable of specifically binding to designated target molecules to form target probe/target molecule complexes; and
(b) detecting the target probe/target molecule complexes.

23. The method of claim 22 wherein detecting the probe/target complexes includes (i) electrophoretically transporting a probe solution including visualization probes through the liquid permeable layer, wherein a given visualization probe is capable of specifically binding to a target probe/target molecule complex to form a bound visualization probe; and (ii) detecting the bound visualization probe.

24. The method of claim 22 wherein detecting the probe/target complexes includes (i) electrophoretically transporting a probe solution including labeled target molecules through the liquid permeable layer, wherein the labeled target molecules are capable of specifically binding to complementary target probes to form labeled target molecule/target probe complexes; and (ii) detecting the labeled target molecule/target probe complexes.

25. The method of claim 22 wherein the one or more target molecules are nucleic acids which have been purified prior to introduction into the liquid permeable layer.

26. The method of claim 22 wherein electrophoretically transporting the sample solution through a liquid permeable layer comprises applying a current of about 50 to 100 microamperes to the liquid permeable layer.

27. A method of detecting a target molecule in a sample comprising:
(a) introducing a first low conductivity buffer solution including the sample into a liquid chamber;
(b) electrophoretically transporting the first low conductivity buffer solution through a liquid permeable layer which is in fluid connection with the liquid chamber; wherein the liquid permeable layer includes at least one microregion having a plurality of microbeads embedded in the liquid permeable layer; the microbeads having a plurality of a target probe on their surfaces; the target probe being capable of specifically binding to the target molecule to form a target molecule/target probe complex;
(c) introducing a second low conductivity buffer solution into the liquid chamber; wherein the second low conductivity buffer solution includes a fluorescently labeled target molecule;
(d) electrophoretically transporting the second low conductivity buffer solution through the liquid permeable layer to form a fluorescent target molecule/target probe complex; and
(e) detecting the fluorescent target molecule/target probe complex.

28. The method of claim 27 wherein the first and second low conductivity buffer solutions are mixed together prior to being electrophoretically transported through the liquid permeable layer.

29. A kit for the analysis of biological samples comprising:
(a) a microarray device comprising a liquid permeable layer including a plurality of microregions, each microregion including a plurality of microbeads embedded in the liquid permeable layer; wherein the microbeads have a plurality of target probes on their surfaces and the microbeads in a given microregion have a plurality of the target probes on their surfaces;
(b) a low conductivity buffer solution; and
(c) a buffer solution including a set of visualization probes.

30. The kit of claim 29 wherein the low conductivity buffer has a conductivity of about 5 to 50 μS/cm.

31. The kit of claim 29 wherein the low conductivity buffer has an inorganic salt content of no more than about 10 mM.

32. The kit of claim 29 wherein the low conductivity buffer includes lysine or histidine.

33. The kit of claim 29 wherein the low conductivity buffer includes barbituric acid, barbital, or a mixture thereof.

34. The kit of claim 29 wherein the visualization probes include fluorescent-labeled target molecules.

35. The kit of claim 34 wherein the target probes are capable of complementary binding to specific nucleic acid target molecules and the visualization probes include fluorescent-labeled nucleic acids capable of hybridizing to one of the specific nucleic acid target molecules.

36. A microarray device for the analysis of biological samples comprising:
a liquid permeable layer including at least one microregion which includes a plurality of microbeads embedded in the liquid permeable layer; wherein the microbeads have a plurality of target probes on their surfaces.

37. The device of claim 36 wherein the liquid permeable layer includes at least 10 of the microregions and a low conductivity buffer having a conductivity of no more than about 50 μS/cm; each microregion having a maximum dimension of no more than about 10 microns; and the microbeads are about 50 to 200 nm in size and all the microbeads in a given microregion have a plurality of a single target probe on their surfaces.

38. A method of detecting a target molecule in a sample comprising:
(a) introducing a plurality of a visualization probe into a low conductivity solution including the sample to form a labeled solution; wherein the visualization probe is capable of specifically binding to the target molecule to form a labeled target molecule;
(b) electrophoretically transporting the labeled solution through a liquid permeable layer; wherein the liquid permeable layer includes at least one microregion having a plurality of microbeads embedded in the liquid permeable layer; the microbeads having a plurality of a target probe on their surfaces; wherein the target probe is capable of specifically binding to the labeled target molecule to form a labeled target molecule/target probe complex on a microbead surface; and
(c) detecting the bound labeled target molecule/target probe complex.

39. The method of claim 38 wherein the target molecule is a nucleic acid; the target probe is capable of hybridizing to the target molecule; and the visualization probe is capable of hybridizing to the target molecule.

40. The method of claim 39 wherein the visualization probe is a fluorescent-labeled nucleic acid.
